(19) United States
(12) Patent Application Publication
(10) Pub. No.: US 2016/0058847 A1
(43) Pub. Date: Mar. 3, 2016

(54) IS100- A Highly Effective Enzyme Principle For Use In Dermal Therapeutics, And For Health And Beauty

(71) Applicant: Issar Pharmaceuticals Pvt. Ltd., Hyderabad (IN)

(72) Inventors: Abani K. BHUYAN, Hyderabad (IN); Ram ISANAKA, Warren, NJ (US)

(21) Appl. No.: 14/843,024

(22) Filed: Sep. 2, 2015

Related U.S. Application Data
(60) Provisional application No. 62/045,129, filed on Sep. 3, 2014.

(51) Int. Cl. A61K 38/48; A61K 8/64; A61K 38/16; C12N 9/50; A61K 9/06

(52) U.S. Cl. CPC A61K 38/48; C12N 9/63; A61K 9/06; A61K 38/168; A61K 8/645; A61Q 19/00

(57) ABSTRACT

A aqueous preparation called IS 100 consisting of at least a hundred of proteins prepared from wheat germ lysate is provided. The preparation shows protease activity and is considerably stable. None of the constituent proteins in IS 100 can permeate synthetic or biological membranes and therefore IS 100 has a variety of in vitro or external applications that require cleaving and cleansing of unwanted or dead tissues on dermal surface.

### PRIORITY

This application claims priority of the U.S. provisional application No. 62/045,129 filed on Sep. 3 2014, the contents of which is incorporated herein by reference.

### FIELD OF THE INVENTION

The present invention relates to a composition and methods for dermal therapeutics and cosmetic use.

### BACKGROUND OF THE INVENTION

There are various cosmetic and therapeutic preparations available that contain various natural ingredients. One such preparation is wheat germ oil. The oil is high in vitamin E and fatty acids and therefore wheat germ oil is commonly used as natural antioxidant, and used as topical application to promote skin health and repair from sun damage as well as relieve of symptoms of dermatitis.

U.S. Pat. No. 7,205,012 discloses use of wheat germ oil as an ingredient in scar healing preparation among other plant oils.

U.S. Pat. No. 6,045,779 discloses use of wheat germ oil as an ingredient in an aerosol for treating hair and skin among other plant oils.

While the oil component of wheat germ has been used topically for skin health, the water soluble part of the germ has not been suggested for dermatological or other therapeutic uses.

Other known uses for wheat germ are nutritional uses. Wheat germ contains a number of nutrients, such as iron, magnesium and zinc. In addition to the high vitamin E content, these nutrients make wheat germ to be considered as a healthy addition to one's daily diet. Wheat germ has been reported to be beneficial to colon health and useful for weight loss.

Topical debriding agents are chemicals that are used locally to clean an open wound by removing foreign material and dead tissue, so that the wound heals without increased risk of infection. This makes the healing faster. Topical drug products containing papain have been used as debriding ointments for removal of dead or contaminated tissue in acute and chronic lesions, such as diabetic ulcers, pressure ulcers, varicose ulcers, and traumatic infected wounds. These products combine papain with other active ingredients, such as urea, chlorophyllin copper complex, and copper sodium chlorophyllin, which are intended to promote removal of unhealthy skin tissue, control local inflammation, reduce wound odors, and rehydrate skin. However, papain containing drugs have been found to potentially produce harmful or near fatal effects including hypersensitivity resulting in anaphylactic reactions. Such cases have required emergency rooms visits, some requiring treatment with epinephrine. The fact that papain powder has been widely used as meat tenderizer illustrates the strength of papain as proteinase.

As papain is known to have potentially harmful effects and a known use of it in dermatological applications requires combination of papain with other ingredients, there is a need for safe natural products for skin treatment.

A ribosomal protein S4 has been described from prokaryotes as well as from eukaryotes. The role of this protein in eukaryotes is not very well understood, however its deficiency in human is thought to produce Turner syndrome. Yadaiah et al. 2012 isolated and cloned a gene encoding S4-protein from wheat seedlings and expressed the gene in Escherichia coli. They identified the protein expressed and isolated from in E. coli cells to have papain-like cysteine protease activity.

There is a constant need for therapeutic preparations for improving skin health, advancing skin beauty and accelerating skin healing after various dermal conditions and wounds. This disclosure provides solutions that none of the previously known art has addressed or suggested.

### SUMMARY OF THE INVENTION

The present invention provides a protein preparation isolated from wheat germ lysate, comprising about 100 proteins and having a protease activity.

The present invention provides a natural protein preparation for dermal therapeutics.

The present invention provides a water soluble protein preparation preferably in form of cream for therapeutic purposes to heal wound tissue, and/or for cosmetic purposes to make the skin shiny and transparent.

It is an object of this invention to provide a protein preparation obtained by gel filtration of wheat germ lysate followed by precipitation of the resulting elute by salt treatment, when the preparation comprises about 100 proteins and has a protease activity.

It is an object of this invention to provide a stable water soluble protein preparation isolated from wheat germ lysate and comprising at least one hundred proteins for dermal therapeutics.

It is a further object of this invention to provide a protein preparation obtained by gel filtration of wheat germ lysate followed by precipitation of resulting elute by salt treatment, wherein the preparation comprises about 100 proteins and has an protease activity and the maximal activity at about pH 7 and at about 40° C.

It is an object of this invention to provide a stable product with natural ingredients for dermal use in thermal therapeutics and for beauty.

It is an object to provide an alternative product to replace the currently known accuzymes.

It is yet another object of this invention to provide a safe method to remove dead dermal tissue and to improve wound healing.

It is still another object of this invention to provide a safe method to make the skin shiny and transparent.

It is a further object of this invention to provide a natural protein preparation made of wheat germ lysate for dermal care.

It is an object of this invention to provide a natural protein preparation made of wheat germ lysate for healing of wounds.

It is still another object of this invention to provide a cream. Lotion, spray, foam, aerosol or cosmetic preparation for application to skin.

### BRIEF DESCRIPTION OF THE DRAWINGS

FIG. 1 shows some parameters for the general activity of formulation IS 100. Rates of hydrolysis of a synthetic peptide Ac-DEVD↓-AFC (N-acetyl-Asp-Glu-Val-Asp-7-amino-4-trifluoromethylcoumarin) as a function of variables are plotted. Note that the rate, ΔF, is determined from the initial slope of the time-dependent fluorescence trace.

(A) The increase in fluorescence due to enzymatic hydrolysis of Ac-DEVD↓-AFC by IS 100 at 37° C. The increase in fluorescence with time is due to the AFC group cleaved by IS 100.

(B) The optimum pH value for hydrolytic activity is ˜7, which is near the physiological pH.

(C) Temperature effect. The preparation has maximal enzyme activity at ˜40° C., close to body temperature.

(D) The enzyme activity is highly sensitive to NaCl concentration.

(E) Inhibition by ZnCl₂. The apparent binding constant, K_{Zn}^{app}=0.31 mM.

(F) Effect of Mg²⁺ ion. The activity passes through a minimum centered at ˜0.1 mM MgCl₂.

FIG. 2 illustrates proteolytic cleavage by formulation IS 100 shown by Western analysis of cleavage products of substrate proteins. IS 100 was incubated with the test protein in the presence and absence of the synthetic inhibitor Ac-DEVD-CHO, and the reaction product was immunoblotted using antibody for the test protein.

(A) and (B), Cleavage of human recombinant PKR and PERK, respectively. Both proteins are cleaved by IS 100 (lane 2), but the cleavage is blocked by the inhibitor Ac-DEVD-CHO which inhibits the IS 100 activity (lane 1). In controls, PKR and PERK (lane 3) were individually incubated in buffer. The IS 100 preparation alone was also run in each case (lane 4). Because the proteins present in IS 100 are not immunoblotted, none shows up.

FIG. 3 shows Ac-DEVD↓-AFC hydrolysis by formulation IS 100 measured in 20 mM HEPES, 0.05% CHAPS, 5% sucrose, pH 7, 37° C.

(A) Effect of various enzyme inhibitors on Ac-DEVD↓-AFC hydrolysis. Iodoacetic acid (IAA) completely inhibits the activity.

(B) IS 100 activity in the presence leupeptin, an enzyme inhibitor.

(C) PMSF inhibits the activity of IS 100 only marginally, and that too requires ˜1000 μM (or 1 mM) of inhibitor concentration.

FIG. 4 shows a typical activity assay: cleavage of Z-FR↓-AMC by IS 100 at ˜32° C. The assay mixture was prepared by combining 400 μL of the IS 100 preparation with 2 μL of Z-FR↓-AMC. Fluorescence was measured immediately after mixing the two.

### DETAILED DESCRIPTION OF THE INVENTION

In this disclosure an active enzyme principle and method to prepare it is provided. The preparation is called IS 100 and it is prepared from wheat germ lysate. IS 100 contains more than a hundred proteins and surprisingly it was found to have a proteolytic activity. The proteolytic activity of the lysate is likely to be due to S4. By its nature S4 itself is so highly active that an isolated and purified form of this protein could not have therapeutic uses, because it may act undesirably fast and would harm healthy cells. Knowing how papain, which is similarly proteolytic as is S4 isolated from wheat seedlings, may have harmful effect, the authors did not consider purifying S4 for therapeutic purposes. Furthermore, isolating and purifying the enzyme would not only be time-consuming and tedious, but mixing with other components to the purified enzyme may add non-natural features to the final product. The added components might even affect the desired enzymatic function.

The inventors unexpectedly found that these problems can be solved by providing a formulation that comprises mixture of over 100 proteins including S4. The mixture of proteins naturally exist in wheat germ and the formulation is made from wheat germ lysate. It was surprisingly found that due to the presence of a large number of proteins in the mixture, S4 does not possess its harmful effects. Therefore, the formulation IS 100 can be used to achieve the objective of cutting, cleaving and chopping of peptides and proteins in therapeutic, and cosmetic purposes.

Since none of the proteins in IS100 formulation can penetrate the cell membrane, and none other than S4 has known enzymatic or otherwise any harmful effect on skin surface, the best way to exploit the proteolytic effect of S4 in therapeutics is to use the IS 100 itself. One may note that the set of activity optima, including pH and temperature, changes little whether S4 is in isolation or in conjunction with other proteins in the IS 100 formulation. Furthermore, the stability of S4 is expected to be better when it is part of the naturally occurring wheat protein group rather than by itself, therefore IS 100 formulation does not need any added stabilizers.

The IS 100 principle of this invention cleaves the peptide bonds in proteins, often several peptide bonds in the protein molecule, to produce fragments of the substrate protein. This can be shown by electrophoretic analysis of a mixture of a test protein and the IS 100 principle. In test tube experiments, the enzyme activity of IS 100 principle can be shown by using a synthetic peptide substrates designed to have a fluorescence group which is not fluorescent if it is not enzymatically cleaved and separated from the substrate. When the fluorescent group is cleaved out of the peptide substrate through the action of IS 100, it becomes highly fluorescent. The emitted fluorescence can thus be used to demonstrate the enzyme activity of IS 100. For example, the dipeptide Ac-DEVD↓-AFC (N-acetyl-Asp-Glu-Val-Asp-7-amino-4-trifluoromethylcoumarin), where the arrow indicates the site of cleavage, is a synthetic substrate for the IS 100 principle. In the following reaction

IS100+Ac-DEVD-AFC→IS100+Ac-DEVD+AFC

the substrate is cut by cleaving the bond between DEVD and AFC. Whereas Ac-DEVD-AFC does not fluoresce as such, the released AFC group through the action of IS 100 is highly fluorescent. The fluorescence emitted by AFC (at 500 nm of light) can be monitored by using a fluorometer to convince oneself that IS 100 indeed has cleaved the bond between DEVD and AFC. Notice that IS 100 can now take part in another round of cleavage of Ac-DEVD-AFC.

The invention is now described in light of examples that are not meant to be limiting.

### Example I

### Preparation of Formulation of IS100

Material used to prepare IS 100 formulation are shown in Table 1.

TABLE 1

| Materials | Company | Product/Cat# |
|---|---|---|
| Wheat Germ | SIGMA | W0125 |
| HEPES | SIGMA | H3375 |
| EDTA | SRL (Sisco) | 054448 |
| CHAPS | SIGMA | C5070 |
| Sucrose | Qualigens | 28105 |
| Sodium chloride | SRL (sisco) | 1940103 |
| Dithiothreitol (DTT) | SIGMA | D0632 |
| Potassium acetate | Qualigens | 26495 |
| Magnesium acetate | MERCK | 105819 |
| Calcium chloride | MERCK | 105819 |
| PMSF | SIGMA | P7626 |
| Z-FR-AMC•HCl | ENZO Life | 260-131-M005 |
| (N-acetyl-Asp-Glu-Val-Asp-7- | Sciences | |
| amino-4-trifluoromethylcoumarin) | | |
| Ac-DEVD-AFC | CALBIOCHEM | 264150 |
| (N-acetyl-Leu-Glu-His-Asp-7- | | |
| amino-4-trifluoromethylcoumarin) | | |
| Ac-LEHD-AFC | CALBIOCHEM | 218765 |
| (Z-FR↓-AMC (N-CBZ-Phe- | | |
| Arg-aminomethylcoumarin) | | |
| Sephadex G25 | GE Health Care | 17-0033-01 |
| Sephadex G100 | GE Health Care | 17-0060-01* |

Two buffers are needed

Buffer A: 40 mM HEPES
100 mM potassium acetate
1 mM magnesium acetate
2 mM calcium chloride
2 mM DTT
Final pH is adjusted to 7.5 by adding concentrated NaOH. Added now is 30 μL of 100 mM PMSF (prepared in isopropanol) per 40 mL of the buffer

Buffer B: 20 mM HEPES
1 mM EDTA
0.05% CHAPS
5% sucrose
20 mM NaCl
1 mM DTT
pH is finally adjusted to 7.5 by adding concentrated NaOH

The following qualitative preparation is carried out in an air-conditioned laboratory maintained at 25° C.:

About 25 g wheat germ was floated in about 40 mL liquid nitrogen in a porcelain mortar and ground by using a pestle. Hand grinding is better done by holding the pestle in the first and driving it circularly on the inside surface of the mortar for about 2 minutes. This is a lab practice for small-scale preparation. Best result is achieved by liquid nitrogen-floating and grinding repeatedly (×4 or 5). The finely ground wheat germ was divided equally and transferred to two centrifuge tubes. Each half of the ground germ was combined with about 10-12 mL of buffer A, already cooled down to ˜4 or 5° C., and made into a baby food-like paste or the consistency of baby food. Adding too much of buffer A to the ground wheat germ should be avoided, because one tries to minimize the volume of the material to be loaded later onto the Sephadex column. The wheat germ paste in then homogenized in a motored homogenizer by inserting the pestle probe into the centrifuged tube. Such powered homogenization may take about a minute or two for each half of the wheat germ paste. The amount of wheat germ paste in different tubes was adjusted for balancing the weights of tubes placed oppositely in the centrifuge rotor. The paste was then centrifuged at ˜14000 rpm (RCF˜15000 g) for 15 minutes at ˜4° C. The supernatant was removed carefully so as to avoid the fat-like float, and the precipitate was discarded. The supernatant was centrifuged again at ˜14000 rpm for 15 minutes at 4° C., and any precipitate obtained was discarded.

The supernatant was loaded onto a 100 mL Sephadex G25 column (the column volume is measured from πr²h where r and h are radius and height of the column in cm) pre-equilibrated in Buffer B. This step may be carried out in the air-conditioned lab where the temperature is ˜22-28° C.). After ˜35 mL elution using Buffer B, a milk-like (but relatively dull, at times even light straw colored) substance elutes which is collected using a fraction collector or manually.

The elute from the Sephadex G25 column was pulled into a measuring cylinder to determine the total volume. In fact, one can collect as much as 50 mL from the Sephadex G25 column. The elute was then subjected to 40% ammonium sulfate cut. The amount of ammonium sulfate needed can be quickly determined from the thumb-rule proportion of ‘1 Liter requires 40 g ammonium sulfate’. The required amount of solid ammonium sulfate can be weighed into a beaker. Ammonium sulfate is added in small amount with a spatula while stirring the solution constantly on a magnetic stirrer. The addition may be repeated every minute. Addition of lumps of ammonium sulfate should be avoided. Lumps, if found, can be gently ground into fine with the help of the steel spatula used for addition of ammonium sulfate. The final salted out or precipitated material contains the IS 100. The solution is then centrifuged at ˜14000 rpm for ˜15 minutes, and the supernatant is discarded.

The pellet is suspended and solubilized in ˜20 mL of Buffer B, and spun again at ˜14000 rpm for ˜15 minutes. In the steps beyond the 40% ammonium sulfate cut, DTT may also be excluded from Buffer B. The other components of this buffer including pH remain the same. The supernatant is then loaded onto a 100 mL Sephadex G150 (or Sephadex G100) column pre-equilibrated in buffer B. After ˜35 mL of elution, the milk-like white elute is collected to a total volume of about 50 mL. This substance is called IS 100.

IS 100 contains at least 100 proteins, which can be observed in SDS-PAGE. One of these proteins is the active enzyme which renders the preparation proteolytic activity. One may still like to check on the activity of the IS 100 preparation as it is or even by diluting into any aqueous medium preferably buffered at pH 7-7.4.

### Example II

### Testing Activity of S100

Fluorogenic peptide substrates like Ac-DEVD↓-AFC (N-acetyl-Asp-Glu-Val-Asp-7-amino-4-trifluoromethylcoumarin), Ac-LEHD↓-AFC (N-acetyl-Leu-Glu-His-Asp-7-amino-4-trifluoromethylcoumarin), and Z-FR↓-AMC (N-CBZ-Phe-Arg-aminomethylcoumarin) can be used to assay the protease activity of IS 100. All three peptide derivatives are cysteine protease substrate analogues. In these peptide sequences the arrow indicates the site of cleavage by IS 100. In a typical assay, ˜100 or 200 μL of IS 100 is combined with 20 mM HEPES buffer, pH 7.4, and ˜2 μL of Z-FR↓-AMC in a quartz cuvette, and time base fluorescence is measured using the following spectral parameters.

Excitation wavelength: 360 nm (1 nm slit)

Emission wavelength: 460 nm (9 nm slit)

Increase in fluorescence with time indicates hydrolysis of Z-FR↓-AMC. It is the released AMC group whose fluorescence emission is measured at 460 nm. The increase in fluorescence can be monitored for a long time (limited by availability of the substrate), but 5 to 10 minutes are sufficient. To convince oneself that the fluorescence is indeed due to Z-FR↓-AMC hydrolysis, a control sample that contains no substrate can be run. The assay procedure for Ac-DEVD↓-AFC or Ac--LEHD↓-AFC hydrolysis is the same except that fluorescence excitation and emission wavelengths are set to 400 and 500 nm, respectively. The enzyme activity assay can be done at any temperature, preferably near the physiological temperature of 37° C.

FIG. 4 shows a typical example of activity measurement.

### Example III

### Preparation of Formulation of IS100 in a Larger Scale

Table 2 below shows the quantities of chemicals required for preparing 1 liter of Formulation IS 100.

TABLE 2

| Materials | Quantity | |
|---|---|---|
| Wheat Germ | 400-700 | g |
| HEPES | 80-100 | g |
| EDTA | 5-10 | g |
| CHAPS | 6-10 | g |
| Sucrose | 700-1000 | g |
| Sodium chloride | 15-20 | g |
| Dithiothreitol (DTT) | 2-5 | g |
| Potassium acetate | 0.1-0.6 | g |
| Magnessium acetate | 0.01-0.80 | g |
| Calcium chloride | 0.01-0.60 | g |
| Ammonium sulfate | about 500 | g |
| PMSF | 0.02-0.80 | g |
| (phenylmethanesulfonyl fluoride) | | |
| Isopropanol (HPLC grade) | 1-5 | mL |
| *Z-FR-AMC•HCl | 1-5 | mg |
| *Ac-DEVD-AFC | | |
| *Ac-LEHD-AFC | | |
| DMSO (Dimethyl sulfoxide) | 1-4 | mL |
| **Sephadex G25 | 300-500 | g |
| **Sephadex G100 | 100-350 | g |

(Note: The peptide substrates marked by * may be used to ensure that the final preparation is enzymatically potent. This check is actually not needed, because the preparation is invariably always active. Nevertheless, the activity is determined by checking the degree of cleavage of one of these peptide substrates. Since Z-FR-AMC.HCl is a much better substrate for IS 100, Ac-DEVD-AFC and Ac-LEHD-AFC need not be considered. Sephadex beads for gel filtration can be reused over and again until they age or are soiled somehow. Thus, if the material necessary for making 1 Liter prep is divided equally into say two halves before gel filtration, then the column can be run twice, which means 200 g of Sephadex G25 and 100 g of Sephadex G100 may suffice).

The formulation is prepared principally in same manner as described in Example I above.

### Example IV

### Cleavage of a Synthetic Peptide Called Ac-DEVD↓-AFC (N-Acetyl-Asp-Glu-Val-Asp-7-Amino-4-Trifluoromethylcoumarin) by IS100

Ac-DEVD↓-AFC is a commercially available synthetic peptide substrate marketed by Calbiochem (product #264150). This substrate is widely used to show that a test protein or a protein preparation that has one or more enzymes can actively cleave the bond between DEVD and AFC (shown by the arrow in Ac-DEVD↓-AFC). A solution of Ac-DEVD↓-AFC is fluorescence-silent as such, but when the bond between DEVD and AFC is cleaved by an enzyme the released AFC group becomes highly fluorescent when excited by using light at 400 nm wavelength. The emitted fluorescence of AFC is read at 500 nm wavelength of light by using a fluorometer to determine whether the enzyme principle is active or not, and if yes, to what extent.

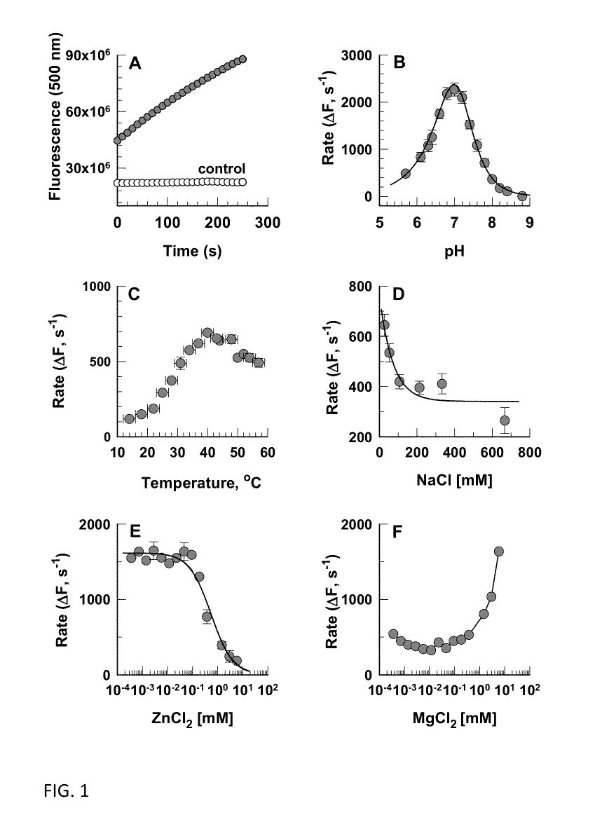

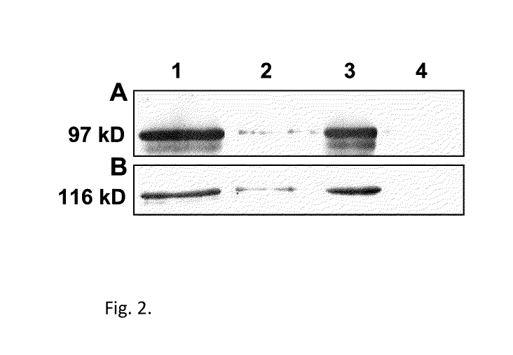

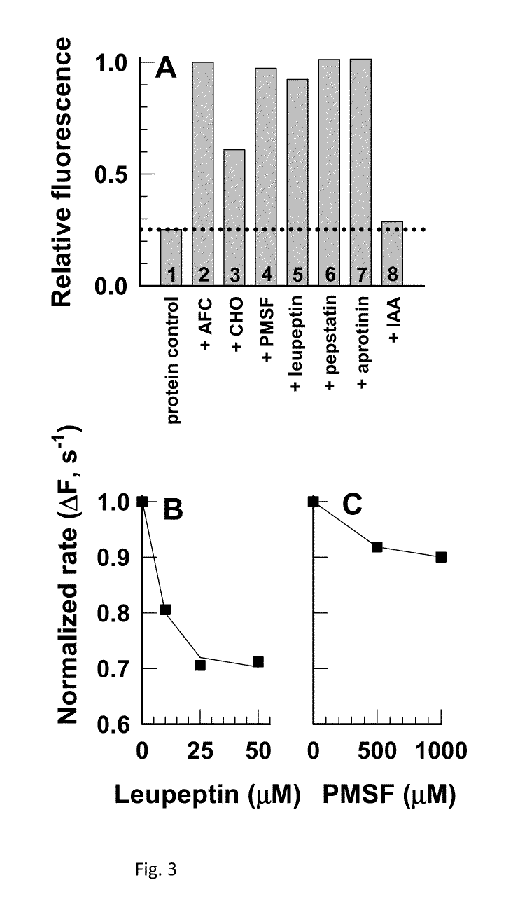

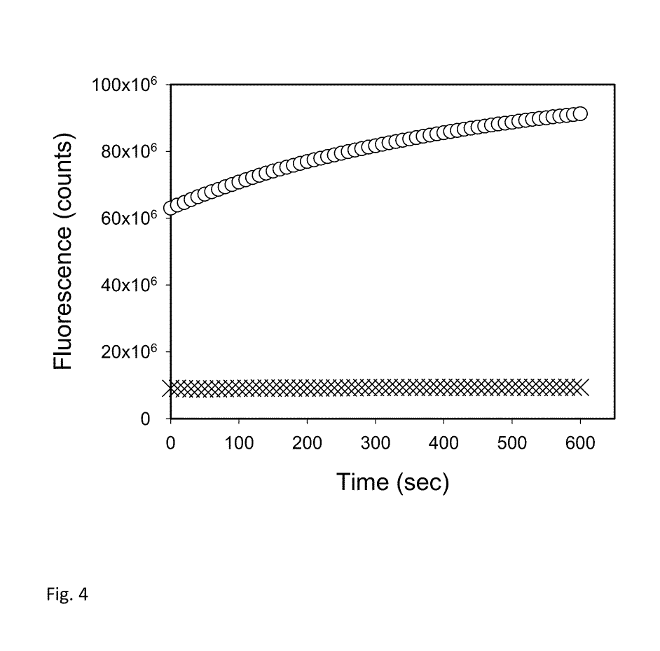

In the present test, 400 μL of IS 100 is combined with 2 μL of a 1 mM solution of Ac-DEVD↓-AFC, and the fluorescence is monitored as a function of time at 37° C. (FIG. 1A). In the control run, where IS 100 is not mixed with Ac-DEVD↓-AFC, there is no fluorescence, which suggests that IS 100 indeed cleaves the substrate, and hence it is an enzymatic principle. This simple experiment can be done under different conditions to find out the optimum values of pH and temperature at which IS 100 works best. FIGS. 1B, and 1C show that optimum pH and temperature for cleavage activity by IS 100 is fairly close to physiological values. Results for the effect of sodium chloride, zinc chloride, and magnesium chloride on the cleavage activity of IS 100 are also shown in panels D, E, and F, respectively, of FIG. 1.

### Example V

### Cleavage of Real Proteins by IS 100

To demonstrate the proteolytic activity toward real proteins, cleavages of recombinant human PKR (double-stranded RNA-activated protein kinase) and PERK (pancreatic endoplasmic-resident eIF2α kinase) are shown below. These two proteins were chosen simply because of their ready availability with the investigators. In fact, any protein will be a substrate for IS 100. In a typical protocol, IS 100 is incubated with the test substrate protein in the presence and absence of the synthetic inhibitor Ac-DEVD-CHO, and the reaction products are analyzed by electrophoresis, the amount of the uncut test protein is immunoblotted with human anti-PKR or anti-PERK antibodies. Note that immunoblotting of a test protein by using the corresponding antibody will show the status of that protein alone, and not other proteins present in IS 100.

In the present case, both PKR and PERK proteins are cleaved by IS 100 (FIG. 2, lane 2), because the immunoblotted gel shows that only trace quantities of these two proteins are left over (lane 2). If the synthetic inhibitor Ac-DEVD-CHO is used to block the proteolytic activity of IS 100 the test protein will remain uncut. Lane 1 in the Figure below shows that both PKR and PERK are not cleaved when the Ac-DEVD-CHO is included in the reactions mixture. In control experiments where PKR and PERK are incubated without IS 100 and analyzed later by immunoblotting using the respective antibodies, one notices no proteolytic cleavage of these proteins, suggesting that all cuts and cleavages (as observed in Lane 2) are solely due to the IS 100 principle. The IS 100 principle was also incubated and electrophoresed. But, since this was not immunoblotted none of the IS 100 proteins show up in the Western blot gel (Lane 4).

### Example VI

### Effects of Protease Inhibitors on the Enzymatic Activity of IS 100

As described above, the proteolytic activity of IS 100 can be blocked by using an inhibitor which blocks the active site of the protein enzyme present in the IS 100 principle. In FIG. 2 inhibitor Ac-DEVD-CHO inhibits the activity of IS 100. Several other chemicals or compounds can be tested for their efficacy in blocking the activity of IS 100. In such experiments, IS 100 is mixed with its synthetic substrate Ac-DEVD↓-AFC, and a constant volume of the inhibitor compound is added to check the extent to which the inhibitor blocks the activity of IS 100. In all experimental runs the concentrations of IS 100 and Ac-DEVD↓-AFC are maintained uniformly. The pH for all experiments is kept at 7 and the experiments are run at 37° C.

The results show that while Ac-DEVD-CHO inhibits the activity of IS 100 significantly (compare bars 1 and 3 in FIG. 3A), iodoacetic acid (IAA) almost completely knocks out the activity (compare bars 1 and 8 in FIG. 3A). Bar 1 in this figure refers to the control where only IS 100 is present—no substrate or inhibitor is added. Leupeptin, an enzyme inhibitor, influences the activity to some extent (bar 5), and the sign of inhibition is also apparent in the presence of PMSF (phenylmethanesulfonyl fluoride), another enzyme inhibitor (bar 4). Profiles for the activity of IS 100 in the presence of different concentrations of leupeptin and PMSF show that the effect due to the former reaches the saturation level (30% inhibition) at ˜25 μM (FIG. 3B), while the effect of the latter is marginal (˜10% inhibition) even at 1000 μM concentration.

### Example VII

### IS 100 is Stable for at Least a Month in Room Temperature

The stability of a preparation of IS 100 obtained in aqueous medium was tested and the current results show that is certainly stable at ˜25-30° C. for at least thirty days. However, preliminary experiments show that the stability will not be lost even after six months. The stability can be determined easily at any point of time by checking the cleavage of Ac-DEVD↓-AFC or Z-FR↓-AMC (N-CBZ-Phe-Arg-aminomethylcoumarin). If no cleavage is found, the preparation is no better. To note, inclusion of glycerol like stabilizers and lowering the storage temperature will prolong the stability.

### Example VIII

### Applications of IS 100

IS 100 formulation can be adopted for any application involving cleaving, chipping, tenderizing, and cleaning of extracellular/external proteins, protein masses, and dead cells and tissues. IS 100 is useful in externally applicable formulations for cleansing dead tissues on skin surface that may accumulate following wound healing, inclement weather conditions (cold weather), or similar situations. After all, dead tissues on dermal surface are mass of inactive proteins which would serve as substrates when presented to IS 100. Because IS 100 is an enzyme principle, it can act upon these proteins to chop them into pieces, thus cleaning or scavenging the dermal surface. Due to the fact that IS 100 contains a large amount of proteins other than the protease, it is not harmful as would the protease alone be. IS 100 may also be used for maintenance of general health (look and glow). It may be used as a substitute for commercially available accuzymes, which are often aggressive to dermal health, and are hence not recommended for prolonged use. In this sense, IS 100 may find its way to therapeutics, health, and beauty depending on how one finds a strategy to administer it.

The basic experiment toward the therapeutic use of IS 100 may involve a general external administration of the preparation diluted into 10 mM phosphate buffer, pH 7.4, on skin surface already scarred and containing dead tissues. Alterations of different skin parameters, including skin color and texture, can be monitored by photography and microscopy. The preparation may be applied on skin once a day or multiple times per day. The preparation may be provided as a cream but other forms may also be used, such as liquid formulas, foams, or aerosols.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A stable water soluble protein preparation having protease activity, said preparation being isolated from wheat germ lysate and comprising about one hundred proteins.

2. The preparation of claim 1, wherein the preparation is obtained by gel filtration of wheat germ lysate followed by precipitation of resulting elute.

3. The preparation of claim 2, wherein the precipitation is obtained by salt treatment.

4. The protein preparation of claim 1 for dermal therapeutics and cosmetic use.

5. The preparation of claim 4 for wound healing.

6. The preparation of claim 4 for making skin shiny and transparent.

7. The preparation of claim 4, wherein the preparation is in a form of a cream.

8. The preparation of claim 1, wherein the preparation has protease activity and maximal activity is at about pH 7 and at about 40° C.

9. The preparation of claim 1, wherein the preparation is provided as a cream, lotion, spray, or foam.

10. A method to remove dead cell material from skin or to improve wound healing, said method comprising applying preparation of claim 1 onto the skin or healing tissue.

11. The method of claim 8, wherein the preparation is a cream.

12. A cosmetic preparation comprising a stable water soluble protein preparation with protease activity, wherein the protein preparation is isolated from wheat germ lysate and comprising about one hundred proteins.

13. The preparation of claim 12, wherein the preparation makes skin shiny and transparent.

14. The preparation of claim 12, wherein the preparation is a cream.
